(19) United States
(12) Patent Application Publication
(10) Pub. No.: US 2008/0103457 A1
(43) Pub. Date: May 1, 2008

(54) Method and System for Treating Heart Failure

(75) Inventors: Gladwin S. Das, Arden Hills, MN (US); Nezam Haider, Irvine, CA (US)

Correspondence Address: INTELLECTUAL PROPERTY GROUP;FREDRIKSON & BYRON, P.A., 200 SOUTH SIXTH STREET, SUITE 4000, MINNEAPOLIS, MN, 55402, US

(73) Assignee: Heart Failure Technologies, Inc. University Enterprise Laboratories (UEL), St Paul, MN (US)

(21) Appl. No.: 11/813,574

(22) PCT Filed: Jan. 11, 2006
(86) PCT No.: PCT/US06/00789

§ 371 Date: Jul. 9, 2007

Related U.S. Application Data
(60) Provisional application No. 60/642,870, filed on Jan. 11, 2005.

(51) Int. Cl. A61M 25/00; A61K 9/00; A61K 38/16; A61K 38/04; A61P 9/04

(52) U.S. Cl. 604/264; 424/484; 514/12; 514/13

(57) ABSTRACT

A system and method for treating heart failure, including the direct or indirect delivery of growth factors, or select portions thereof, to the heart in a therapeutic manner to provide sustained release and induce cell proliferation. The growth factors include peptide hormones, and further include particular sequences derived from human insulin like growth factor 1 (hIGF-1). The factors can be delivered to the pericardial space in a vehicle such as a hydrogel and using a device described herein.

### TECHNICAL FIELD

In one aspect, the present invention relates to the treatment of heart failure. In another aspect, the invention relates to peptide hormones such as insulin-like growth factor-1 (IGF-1).

### BACKGROUND OF THE INVENTION

Heart failure is the most prevalent cardiovascular disease, which results in a large number of hospital admissions and carries with it an extremely high mortality rate. It is estimated by the U.S. National Institutes of Health (NIH) that 4.8 million Americans have congestive heart failure, calling it a “new epidemic.” That number is projected to double by 2007. On average, 550,000 new cases are diagnosed with the often-fatal condition each year.

By simple definition, heart failure is a chronic ailment where the heart fails to function normally due to impairment of the heart's pumping ability (left ventricular systolic dysfunction). However, what this diagnosis truly represents is a complex clinical syndrome that can develop from virtually any cardiac disorder of the pericardium, myocardium, endocardium, or great vessels, but the majority of heart failure patients have symptoms due to the impairment of the left ventricular function. Damage to the left ventricle of the heart limits the ability of the ventricle to eject blood from the ventricle, resulting in an enlarged, weakened muscle, which can no longer squeeze effectively to pump the blood through the chamber.

Difficulty in breathing (dyspnea), ankle edema, and fatigue affect the patient's ability to perform their normal activities of daily living and are the hallmarks of the clinical manifestations of heart failure. Noticeably, the clinical picture of heart failure worsens to dominate the life of the patient: frequent physician office visits, numerous hospitalizations, multiple medications, with resultant side effects, and activity restrictions impacting on the quality of life for the patient with the consequential functional capacity impairment of the cardiopulmonary system.

The prognosis of end-stage heart failure is dismal. Despite several decades of research, none of the mechanical assist devices and total artificial hearts have found widespread use due to several key technical limitations of these forms of therapy.

In view of the rapidly rising rates of new cases regardless of the differentiation of acute vs. chronic heart failure, treatment is primarily aimed at the underlying cause with concomitant therapy to improve cardiac performance. Therapeutic procedures include, multiple medications such as beta-blockers, diuretics, digoxin, anti-arrhythmics, anticoagulants, implantable defibrillators and biventricular pacing, left ventricular assistance device therapy and cardiac transplantation (with limited availability due to the scarcity of donor hearts).

In developing new forms of therapy the factors that are considered critical are long term efficacy in relieving symptoms and prolonging life of patients, in addition to ease-of-manufacturability, ease-of-use and the ability to provide such treatment on a global scale in centers with basic medical facilities.

On a separate subject, the sequence and activity of peptide hormones such as insulin-like growth factor-b 1 (also referred to as somatomedin C) has been extensively studied and described (1-10). In one aspect, IGF-1 has been shown to improve myocardial function in normal adult rats (11), in rats after myocardial infarction (13), in patients with chronic heart failure (14), and in healthy humans (15).

The potential therapeutic use of IGF-1 has been implicated in cardiac disorders related to growth failure, catabolic states, myocardial infarction, and diabetes (16,17). Experimental evidence indicates that IGF-1 may enhance myocardial contractility directly (18,19,20). IGF-1 causes IP₃ accumulation in rat cardiomyocytes (21), and increases intracellular Ca²⁺concentration, in parallel to its action on myocyte contractility (18,19,20). Although IGF-1 may enhance cardiac contractility through an increase in contractile protein synthesis, several intracellular signaling pathways related to IGF-1 also have been implicated (9)-these include tyro sine kinase, tyro sine kinase phosphatase, PI-3 kinase and protein kinase C (9,22,23,12). Activation of one or more of these intracellular signaling pathways may be directly related to the elevation of intracellular Ca²⁺, and therefore the acute positive myocardial response.

IGF-1 may also increase cardiac contractility without affecting intracellular Ca²⁺concentration, perhaps due to an increase in intracellular Ca²⁺ sensitivity (12). Membrane ion channels have been implicated also in IGF-1-related modulation of cardiac function. IGF-1 stimulates T-type calcium current density in cardiac myocytes by altering gene expression (24). IGF-1 is reported to double dihydropyridine (DHP)-sensitive Ca²⁺ channel activity in cardiac myocytes, possibly via a PKC-dependent mechanism (25). Long-term IGF-1 administration also has been found to regulate cardiac K⁺channel expression in neonatal rat ventricular myocytes. Both calmodulin-dependent kinase and tyrosine kinase have been found to contribute to the IGF-1-mediated increase in cardiac K⁺channel expression (26,27).

IGF-1 may also affect cardiac muscle mass by preventing programmed cell death (8). Apoptosis in cardiomyocytes contributes to the development of heart failure. In a murine model of myocardial ischemia reperfusion, IGF-1 administration decreased myocardial apoptosis (28). In a coronary artery ligation model creating murine myocardial infarction, transgenic over expression of IGF-1 decreased myocardial cell death, and ventricular dilatation (29). In a canine model of heart failure induced by overpacing, IGF-1 reduced the number of apoptotic cardiomyocytes and increased contractile function (8). IGF-1 mediated inhibition of apoptosis has been shown to be associated with the increased expression of a member of the anti-apoptosis family of Bcl-2 proteins (30). IGF-1may act as a survival factor via stimulation of the Bcl-2 family of proteins. Several signaling pathways such as tyrosine kinase, PI3 kinase and MAP kinase have also been suggested tomediate the anti-apoptotic effects of IGF-1 (31, 30). Transgenic over-expression of endogenous IGF-1 provides a more chronic model of hormone exposure. An increase in cardiac myocyte number in vivo was observed when IGF-1 overexpression was restricted to the heart.

In spite of studies and findings such as those described above, there remains an urgent need for new treatment modalities, and ultimately preventive measures or cures, for all cardiovascular disease. In particular, there currently remains an ongoing and strong desire to develop new therapies to alleviate symptoms and prolong survival in patients with heart failure.

### SUMMARY OF THE INVENTION

The present invention provides a system and method for treating heart failure, including by the direct or indirect delivery of growth factors, or functional analogues or portions thereof, to the heart in a therapeutic manner to provide sustained release and induce revitalization and/or proliferation of myocardial cells with improvement or retention of cardiac function. The growth factors include peptide hormones, and further include particular sequences derived from human insulin like growth factor 1 (hIGF-1). The factors can be delivered to the pericardial space in a vehicle such as a hydrogel or infusion using internal and/or external pumps, osmotic pumps, and the like, optionally also including a pericardial access device described herein.

The present invention provides a method and related compositions and system for alleviating, including preventing, symptoms and prolonging survival in patients with or at risk of heart failure. The system includes the delivery of one or more bioactive agents directly or indirectly to the heart, and preferably to the pericardial sac itself. Such bioactive agents are preferably peptide hormones, most preferably comprising human IGF-1, including in its native or modified forms as well as analogues and functional portions thereof. In such an embodiment, the bioactive agent is preferably provided and delivered to the body in a composition (e.g., hydrogel) under conditions suitable to provide desired release kinetics, including prolonged release of the bioactive agent in therapeutic amounts. The method and composition of this invention can be used for “treatment” in a preventive, curative, palliative, supportive, and/or restorative manner with respect to heart failure.

In a preferred embodiment, the method of the present invention comprises the steps of: a) providing a bioactive agent comprising one or more peptide sequences derived from human IGF-1, including analogues and functional portions thereof, b) incorporating the bioactive agent in a stable, releasable fashion into a pharmaceutically acceptable vehicle to provide a deliverable composition, and c) delivering the composition to the heart by intrapericardial delivery (e.g., injection) in a manner sufficient to provide a therapeutic effect upon release of the bioactive agent. In a particularly preferred embodiment, the bioactive agent comprises, a sequence selected from the group consisting of SEQUENCE ID'S 1-5 and analogues thereof, the vehicle comprises a hydrogel, and delivery to the pericardial sac is accomplished by the use of a pericardial access device.

In turn, the present invention provides a system, including related compositions and methods, for the use of bioactive agents such as growth factors, and portions thereof, to improve myocardial performance. Preferred bioactive agents comprise growth factors, and in a particularly preferred embodiment, a family of peptides providing an optimal combination of properties such as biological activity (similar to physiological growth factors) and ease of manufacture and use.

To the best of Applicant's awareness, no products or processes that involve cellular therapy or revitalization therapies have currently been approved for heart failure. Accordingly, the system of the present invention can serve a vital and revolutionary role in therapeutic approaches to congestive heart failure.

### DETAILED DESCRIPTION

A “system” of this invention will typically include one or more bioactive agents in suitable combination with one or more compositions and/or devices adapted to deliver the agents to the heart. In addition to a system as described herein, the present invention provides components used to prepare such a system, several of which are considered novel in their own right, as well as a method of preparing such components, and in turn, a method for preparing the system itself, and a method of using the system to treat, including to prevent, heart failure.

The bioactive agent of this invention preferably comprises a peptide, and more preferably includes an insulin-like growth factor (e.g., IGF-1, IGF-2, or a mixture of both IGF-1 and IGF-2). The IGF may be any IGF of any species. Preferably the IGF is the IGF homologue specific to each species. The IGF may be isolated from a naturally-occurring source, or it may be chemically synthesized or produced by recombinant DNA technology.

It is to be understood that the present invention extends to biologically active fragments or functional analogues of IGF, e.g., to analogues or derivatives of human IGF in which the wild-type IGF sequence includes additions, deletions or substitutions by another amino acid or an amino acid analogue, provided that the biological activity of the IGF is retained. The terms “fragment”, “analogue”, and “derivative” of IGF mean a molecule which retains some or all of the biological function or activity as IGF. Thus an analogue includes a proprotein which can be activated by cleavage of the proprotein portion to produce an active mature polypeptide.

The term “treatment” as used herein is intended to include either therapeutic treatment of heart failure, or preventive or prophylactic procedures performed before the occurrence of the disorder. In accordance with this invention, the IGF is administered in therapeutically effective amounts. The term “therapeutically effective amount” as used herein means that amount necessary at least partly to attain the desired effect, e.g., regeneration and/or preservation of heart tissue. Such amounts will depend on the particular injury being treated, the severity of the injury, and the characteristics of the individual subject, including age, physical condition, size, weight and other concurrent treatment, and will be at the discretion of the attending physician or veterinarian.

Preferably the IGF is administered by localised administration. Such administration may be achieved directly at the site, for example by one or more intrapericardial injections or implants, or with a delivery system. Alternatively, other modes of administration, such as systemic injections, may be used, provided that they increase the amount of IGF within heart tissue to attain the desired effect.

Methods and pharmaceutical carriers for the preparation of pharmaceutical compositions, including compositions for intrapericardial administration, are well known in the art, as set out in textbooks such as Remington's Pharmaceutical Sciences, 18^{th} Edition, Mack Publishing Company, Easton, Pa., USA. Suitable pharmaceutically acceptable carriers and/or diluents include conventional solvents, saline solutions, dispersion media, fillers, aqueous solutions, antibacterial and antifungal agents and absorption-promoting agents. Except insofar as any conventional medium or agent is incompatible with the active ingredient, its use in the pharmaceutical compositions of the present invention is contemplated. Supplementary active ingredients which have the ability to promote healing or to inhibit inflammation may also be incorporated into the compositions. For example, the pharmaceutical composition may additionally include one or more other cytokines, including but not limited to insulin, epidermal growth factor, fibroblast growth factor, betacellulin, transforming growth factor alpha or transforming growth factor beta.

The administrations contemplated by the present invention include administration of any formulations suitable for delivery of IGF, such as aqueous isotonic solutions, suspensions, gels, and polymers impregnated with IGF, or for topical administration of IGF, such as aqueous creams, ointments, gels, lotions, sprays, microspheres, liposomes, wound dressings, and synthetic polymer dressings or sutures impregnated with IGF, and the like.

The bioactive agent of this invention can be delivered in any suitable maimer and using any suitable means, including in vehicles that comprise liquids, solids, semisolids, matricies, powders, and/or particles, and which in turn can be bioabsorbable, biodegradable or stable. Emerging technologies can also be relied upon, including medicated powders pumped into the tissue at supersonic speeds, implanted biochips, and nanomolecular transportation systems. In turn, the vehicle can be delivered to the desired site with or without bioactive agent, and using any suitable means, including by open or minimally invasive access, using catheters, membranes, lasers, or other medical-surgical instruments. Suitable compositions, and corresponding vehicles used to prepare such compositions, provide an optimal combination of advantages such as efficacy, ease of administration and compliance, dosage accuracy and frequency, release kinetics, decreased side effects and cost reduction.

A bioactive agent suitable for use in such a system will typically and preferably include a peptide, more preferably a peptide derived from human IGF-1, and even more preferably, a putative peptide that comprises one or more unique regions of hIGF-1, which are not shared by insulin and IGF-II.

Human insulin-like growth factor I (hIGF-1) is a single chain polypeptide of 70-aminoacids and bears structural homology with proinsulin. It has short-term metabolic effects and long-term effects on cell proliferation and differentiation and promotes cell growth and differentiation of various cell types. IGF-1 is produced in the liver under the control of growth hormone and also in a number of other cell types and acts locally in an autocrine and paracrine manner. The mitogenic activity of IGF-1 is mediated through binding mainly to the IGF-1 receptor which exist in a number of tissues. IGF-1 associates with specific binding proteins (IGFBPs) mainly IGFBP-3 in plasma and tissue which regulates its bioactivity.

hIGF-1 is well characterized both structurally and functionally. Mature protein is a single chain polypeptide of 70-amino acids bears structural homology to proinsulin. It has four domains—B, C, A and D in same order. Domain B spans from 1-29 amino acids followed by region C a loop of 12 amino acids (aa 30-41), which links domain B to domain A (aa 42-62) followed by 8 amino acid long region-D (aa 63-70).

IGF-1 binding to the IGF-I receptor results in autophosphorylation by receptor's intrinsic kinase activity, and tyrosine phosphorylation of members of the insulin-receptor substrate (IRS) family. Tyrosine-phosphorylated IRS-1 and IRS-2 interacts with specific cytoplasmic proteins containing SH2 (src homology 2) domains, leading to the transduction of downstream signals. The IGF-1 signaling cascades are activated by the association of growth receptor binding protein 2-Son of Sevenless (Grb-2-SOS) with phosphorylated Crk and She, resulting in the activation of Ras, and the sequential activation of serine/threonine kinase Raf (A-Raf, B-Raf and c-Raf) and MAP kinase kinase (MEK) which activates MAP kinases (ERK-1 and ERK-2) by phosphorylation. MAP kinases in turn activate a number of transcription factors mediating IGF-1 stimulated of DNA synthesis and mitogenesis. The p85 regulatorysubunit of PI3-kinase is another important SH2 domain-containing protein, the binding of which to tyrosine-phosphorylated IRS-1 activates the catalytic function of the 110-kDa subunit of PI3-kinase. PI3-kinase is essential for the transduction of metabolic growth and functional effects of IGF-1, including stimulation of glucose transport, antilipolysis, protein and glycogen synthesis, inhibition of apoptosis, and more recently, IGF-1 mediated cardiomyocyte contractility.

In heart, IGF-1 has specific cardiac effects as well as general growth and metabolic effects. IGF-1 hormone and IGF-1 receptors are present in fetal and adult myocardium (1,2). It increases cardiac DNA and protein synthesis, reduces protein degradation, and participates in early neonatal cardiomyocyte proliferation and maturation. IGF-1 enhances myofibril development in long-term cultures of adult rat cardiomyocytes (3). IGF-1 increases myocardial DNA and protein synthesis in isolated cardiomyocytes (4,5). IGF-1 is necessary for entry into the S phase of the cell cycle, and IGF-1 has been reported to modulate the induction of genes that regulate the cell cycle (6,7). In transgenic mice overexpressing IGF-1 in myocardium, total heart weight is increased by 50%, and the number of cardiomyocytes is increased by 20-50% (7). IGF-1 is associated with induction of expression of contractile proteins such as actin, myosin light chain-2, troponin I, beta-myosin heavy chain, and skeletal alpha-actin in neonatal rat cardiac myocytes (5,9). The growth promoting actions of IGF-1 are mediated by tyrosine kinase and downstream signaling pathways which involve IRS-1, PI3-kinase and ERK (10).

Sources of IGF-1 are available, for instance, from vendors such as GroPep, Limited, including for instance, an analogue identified as Long™R³IGF-1. See “An Analogue of IGF-1”, Yandell, et al., BioProcess International, pgs 56-64, March 2004. Other forms of IGF-1 suitable for use in the present invention, including sequences and fusion peptides thereof, can be obtained or synthesized using techniques within the skill of those in the art, given the present description. See, for instance, U.S. Pat. Nos. 5,077,276 (Ballard, et al.), 5,164,370 (Ballard, et al.), and 5,330,971 (Wells, et al.).

Preferred IGF-1 peptides for myocardial revitalization therapy are those that employ a minimum of IGF-1 peptide sequences and which have:

1. Superior specificity and higher binding affinity for its cognate receptor than native IGF-1.

2. Receptor occupancy that produces better or similar biological effects on cardiac general growth and metabolism.

3. No substantial binding affinity for the related insulin receptor or for the IGF type 2 receptor, in order to minimize any undesired side effects.

Region C and D are not present in insulin. Although absence of region D has little effect on type I and II IGF receptor binding, its presence sterically hinders IGF-1 binding to the insulin receptor. Absence of domain B does not change affinity for IGF-1 receptor but it loses affinity for the soluble IGF binding proteins. Lack of region A has no effect on the affinity for IGF-1 receptor but it significantly loses affinity for type-2 IGF receptor. Furthermore, site-specific mutation studies identified critical amino acid residues required for efficient IGF-1 binding. Available information indicates region A, B and D are not involved in the high affinity binding of the hIGF-1 to the type 1 IGF receptor.

Without intending to be bound by theory, for these and other reasons, it would appear that peptides spanning over region C is more suitable for use in heart failure therapy. Although inclusion of region D will not change the affinity for IGF-1 receptor, it will greatly decrease binding to insulin receptor and hence significantly reduce side effects.

In hIGF-1 the B (1-29) and A (42-62) regions are linked with a section of 12 amino acids (30-41) termed the C region or the loop. The carboxyl terminus of hIGF-1 contains an eight residue extension to the A region termed the D region. A and B regions of hIGF-1, hIGF-2 and insulin exhibit high degree of sequence homology, so the peptides of the present invention are designed to span the unique regions of IGF-1 and their combinations (Table II). The putative peptides constitute various combinations and permutations (Table I) of the unique regions of hIGF-1, which are not shared by insulin and IGF-2.

TABLE I
“Heart Failure Peptides”

| | | |
|---|---|---|
| Peptide I | | |
| (SEQUENCE ID 1) | BCAD | GFYFNKPTGYGSSSRRAPQTGIVDECCFR |
| | | SCDLRRLEMYCAPLKPAKSA |
| Peptide II | | |
| (SEQUENCE ID 2) | CAD | GYGSSSRRAPQTGIVDECCFRSCDLRRLE |
| | | MYCAPLKPAKSA |
| Peptide III | | |
| (SEQUENCE ID 3) | CD | GYGSSSRRAPQTPLKPAKSA |
| Peptide IV | | |
| (SEQUENCE ID 4) | CDX3 | GYGSSSRRAPQTPLKPAKSAGYGSSSRRA |
| | | PQTPLKPAKSAGYGSSSRRAPQTPLKPAK |
| | | SA |
| Peptide V | | |
| (SEQUENCE ID 5) | DC | PLKPAKSAGYGSSSRRAPQT |

TABLE II
hIGF-1 Structure:

| | | |
|---|---|---|
| Region | AA | Sequence |

| | | |
|---|---|---|
| Signal | | |
| (SEQUENCE ID 6) | 1 . . . 21 | MGKISSLPTQLFKCCFCDFLK |
| Propeptide | | |
| (SEQUENCE ID 7) | 22 . . . 48 | VKMHTMSSSHLFYLALCLLTF |
| | | TSSATA |
| Region-B | | |
| (SEQUENCE ID 8) | 49 . . . 77 | GPETLCGAELVDALQFVCGDR |
| | | GFYFNKPT |
| Region-C | | |
| (SEQUENCE ID 9) | 78 . . . 89 | GYGSSSRRAPQT |
| Region-A | | |
| (SEQUENCE ID 10) | 90 . . . 110 | GIVDECCFRSCDLRRLEMYCA |
| Region-D | | |
| (SEQUENCE ID 11) | 111 . . . 118 | PLKPAKSA |
| Propeptide | | |
| (SEQUENCE ID 12) | 119 . . . 153 | RSVRAQRHTDMPKTQKEVHLK |
| | | NASRGSAGNKNYRM |

Note:
1. Peptide I-BCAD-First 21 amino acids of domain B from amino terminal end of a mature peptide are deleted. The resulting peptide loses affinity for IGF binding proteins, but affinity is not changed for IGF-1 receptors.
2. Noting a minor modification of the peptide I-BCAD, in that the addition of 3 amino acids includes Tyr²⁴.

With regard to peptides III, IV and V—peptides containing region C and D can be synthesized in two different orientations, namely as both CD and DC fusion sequences, as well as CDX3 and as 3 CD tandem repeats, in order to evaluate the best peptide for any particular disease, and corresponding course of the therapy.

Additional preferred peptides, include:
1) Peptide I BCAD—21 amino acids of domain B from amino terminal end of a mature peptide are deleted. Resulting peptide lose affinity for IGF binding proteins, but affinity is not changed for IGF-1 receptors. It also includes Tyr²⁴ as well as amino acid residues between positions 24-37 of the hIGF-1, to improve type 1 receptor interaction.
2) Peptide II—Region C and D is linked by domain A to maintain spatial distance and configuration of native peptide.

Delivery compositions and devices suitable for use in a system of this invention include those that provide an optimal combination of such features as delivery kinetics and controllability, biocompatibility (including inertness, compatability of degradation products), biodegradability, and the ability to be prepared and used under sterile conditions.

Suitable delivery routes include, but are not restricted to, intramuscular, subcutaneous, percutaneous, oral, transdermal, intranasal, ocular, intrapericardial, direct myocardial injection, percutaneously through the left ventricular cavity or surgically through the epicardium, as well as infusion into the pericardial sac using implantable and/or external pumps

Delivery compositions can take any suitable form, e.g., liquid, solid, suspension or emulsion, and combinations or hybrids thereof. For instance, solid delivery compositions can be prepared from a variety of materials, including metallic, ceramic, and polymeric materials, as well as combinations thereof.

Preferred delivery compositions include polymeric biomaterials, including those that permit bioactive delivery to occur by diffusion of agent from the biomaterial, degradation of the biomaterial, and/or swelling of the biomaterial. Examples of suitable polymeric biomaterials include, but are not limited to polyethylene glycols, hydrogels, vinylic based delivery systems, collagen based delivery systems, and injectable putties.

Suitable hydrogels, also known as water-containing gels, are polymers characterized by hydrophilicity and insolubility in water. See, for instance, Hydrogels, pp. 458-459, in “Concise Encyclopedia of Polymer Science and Engineering”, J. Kroschwitz, ed., John Wiley and Sons, 1990, the disclosure of which is incorporated herein by reference. Examples of hydrogels include those described by, and often commercially available from such sources as MacroMed, Inc. (e.g., as the “ReGel” injectable gel depot system), Gel Del Technologies, Inc. (Saint Paul, Minn.), and Controlled Therapeutics (Glasgow, Scotland), and others. A composition can be provided in any suitable form, e.g., as engineered tissue, implants, prostheses, or artificial parts (such as liposomes, nanoparticles, microparticles, microcapsules, microspheres), or any suitable combination thereof. See, for instance, MacroMed's U.S. Pat. No. 6,287,588 (Shih, et al.), the entire disclosure of which is incorporated by reference, which describes a dual phase system that includes both microparticles and a biodegradable gel, in order to provide a release profile that includes both an initial burst and sustained release.

Local delivery of the bioactive agent within the pericardial space can be accomplished in any suitable manner. Suitable delivery compositions and/or devices provide an optimal combination of such properties as compatability between the composition, device and bioactive agent, as well as ease of delivery and use, intramural retention and release kinetics, and the effects of delivery on vascular and tissue structural integrity. A variety of delivery devices (e.g., catheters) are available that can be used for endoluminal-based intramural delivery to the pericardial space. Examples include those employing pressure-driven convective transport of fluid. Examples of suitable catheters include a microporous infusion catheter (MIC; Cordis Corp., Miami, Fla., USA) consisting of a flow-restricting inner balloon with multiple 25 μm holes and an outer balloon membrane with 0.8 μm pores, which provides a “weeping” convective transport of the infused drug during balloon inflation.

More preferably, the invention employs a catheter-based approach to pericardial access. Drug delivery into the pericardial sac differs from endoluminal deliveries by (1) comparatively enhanced consistency, and (2) prolonged exposure of either coronary or myocardial tissues to drug as a result of a reservoir function of the pericardium. See H P Stoll, et al., Clin. Cardiol. Vol. 22 (Suppl. I), I-10-I-16 (1999).

Coupled with the composition in which the bioactive agent is provided, the use of pericardial delivery provides significant advantages and opportunities over the art. Available pericardial devices typically include either a hollow, helical-tipped catheter designed for controlled penetration through the myocardium during fluoroscopic visualization, or a sheathed needle with a suction tip designed for grasping the pericardium and accessing the pericardial space using a transthoracic approach while avoiding myocardial puncture. Suitable pericardial access devices can be provided in any suitable configuration (e.g., as needles, catheters, introducers and the like) and used in any suitable manner, including any suitable, and preferably minimally invasive, access route.

In turn, preferred pericardial deliveries can be performed by either a percutaneous transventricular method, or a transthoracic approach. The transventricular method typically employs a hollow, helical-tipped catheter designed for controlled penetration through the myocardium into the pericardial space during fluoroscopic visualization. Following placement of a sheath into the right carotid artery, a catheter can be placed through the sheath and advanced under fluoroscopic guidance into the left ventricle to the cardiac apex, with the catheter tip directed inferiorly.

Upon firm contact with the myocardium, the catheter tip can be advanced through the myocardium using a gentle turning motion. After advancement over several mm, hand infusion of a suitable dye solution can be initiated and contrast location monitored fluoroscopically. Successful intrapericardial tip placement can be identified by accumulation of contrast in the pericardium, at which point the catheter can be fixed in position and flushed with saline prior to delivery of the desired agent. Following delivery, final catheter position can be confirmed by fluoroscopic visualization of a bolus of air instilled into the pericardial space, after which the catheter can be removed.

By contrast, a transthoracic approach can be used that involves a sheathed needle with a suction tip designed for grasping the pericardium and accessing the pericardial space while avoiding myocardial puncture. Such a device can be placed from a subxiphoid position into the mediastinum under fluoroscopic guidance and positioned onto the anterior outer surface of the pericardial sac. The sac is then retracted under manual suction, entered by the needle, and a guidewire is placed through the needle lumen into the pericardial space. The wire can be advanced several centimeters in order to identify a configuration that reflects intrapericardial position, after which the needle can be removed and a suitable dilator catheter inserted over the wire. Following removal of the wire, successful intrapericardial tip placement can be confirmed by accumulation of infused contrast in the pericardium, at which point the desired agent can be delivered in a suitable volume, after which the catheter itself is removed.

Bioactive agents of this invention can be delivered in any suitable manner in order to revitalize the myocardium, and in turn, improve myocardial performance. Peptides can be delivered directly or indirectly to the myocardium, including by systemic delivery, indirect (e.g., targeted) delivery to the heart, and directly to the heart, e.g., by intrapericardial instillation.

In turn, such bioactive agents can be delivered in a manner that provides release or delivery kinetics of choice, for instance, they can be delivered rapidly or by sustained release, or any suitable combination thereof, including in a manner that provides whatever initial or periodic bursts of release might be desired. In a preferred embodiment, the bioactive agent is delivered in a manner to provide an immediate release, followed by sustained release over time (e.g., days to weeks).

By way of background, the pericardium (pericardial sac) is a conical membranous sac in which the heart and the commencement of the great vessels are contained. The pericardium is fluid-filled and functions to prevent dilation of the chambers of the heart, lubricates the surfaces of the heart, and maintains the heart in a fixed geometric position. It also provides a barrier to the spread of infection from adjacent structures in the chest cavity and prevents surrounding tissue(s) from adhering to the heart. The space between the pericardium and the heart, known as the pericardial space, is normally small in volume and includes the fluid therein.

Intrapericardial delivery, e.g., instillation of the peptide is presently preferred, in that it can be used in a manner that permits slow and sustained release of the agent, and the advantage of high local concentrations without systemic effects. See, for instance, U.S. Patent Application for “Injection of Recombinant Proteins”, Application No. 20040013653 Simons, the disclosure of which is incorporated herein by reference, in which pericardial delivery is accomplished by instillation of the protein-containing solution into the pericardial sac. The pericardium is accessed either via a right atrial puncture, transthoracic puncture or via a direct surgical approach. Once the access is established, the material is infused into the pericardial cavity and the catheter is withdrawn. Alternatively, the delivery is accomplished using slow-release polymers such as heparin-alginate or ethylene vinyl acetate (EVAc). In both cases, once the protein is integrated into the polymer, the desired amount of polymer is inserted under the epicardial fat or secured to the myocardial surface using, for example, sutures. In addition, polymer can be positioned along the adventitial surface of coronary vessels.

Intrapericardial injection of bioactive agents of this invention can be performed in any suitable maimer, and using any suitable device. A preferred device is available under the name PerDUCER™ pericardial access device, available from Comedicus Incorporated, Columbia Heights, Minn. This device uses suction to create a lifted section of the pericardium, called a “bleb.” The bleb, in turn, is secured to an elongated access device by a suction force exerted through a side wall port that is in a plane parallel to the longitudinal access of the device. Once formed, the bleb is punctured by a needle of limited travel that penetrates the bleb in a direction substantially tangential to the epicardial surface of the heart. While creating a bleb by suction through a side wall port combined with a tangential needle approach to the bleb can reduce the chance of puncturing or lacerating the myocardium, accurately penetrating the pericardium at a desired location may be difficult due to the motion of the heart during normal cardiac contraction relative to the orientation of the axial dimension of the device.

In use, an incision of sufficient size for passage of the guide tube of the access device is made in the thoracic wall, for example in the subxiphoid region, using known methods. A second incision can be made for insertion of an endoscope into the thoracic cavity for visualization of the access procedure. Alternatively, the access procedure can be visualized with the aid of known external visualization systems, including, for example, fluoroscopy, ultrasound, etc. In a subxiphoid approach the device is advanced percutaneously through the first incision over the diaphragm into the mediastinal space until the distal end of the device contacts the pericardial surface of the heart.

The access device is aligned at a desired location on the pericardial surface of the heart and suction is applied to the guide tube lumen to form a bleb of pericardial tissue that passes into the guide tube lumen, through the distal port and extending proximal to the shoulders. Once the bleb is formed, the piercing tip of the penetrating body is advanced distally to pierce the bleb. A guidewire is then passed through the guidewire port through the lumen of the penetrating body and into the pericardial space. The device is removed and a catheter or other known material transport tube is guided over the guidewire into the pericardial space. The guide wire can be removed during fluid removal or administration of the desired material into the pericardial space. With a distal end of the material transport tube located in the pericardial space, a proximal end of the material transport tube can be fixed outside the patient's body, using known methods, for long or short term access to the pericardial space through the material transport tube.

The system, including bioactive agents and corresponding compositions, of the present invention are complementary to existing forms of therapy for heart failure, including mechanical devices, electrophysiological forms of therapy, and pharmacological agents. Since the system can be used to revitalize and repair the myocardium, it can be used with most, if not all, patients with overt failure, and can be considered for asymptomatic patients with left ventricular dysfunction as well. The system can be coupled with ease-of-use through minimally invasive techniques. Since the therapy results in myocytes being revitalized, it is expected to become the standard of care.

The following articles are incorporated herein by reference:

1. Haskell J F Tucker D (1994) Binding of insulin-like growth factors (IGF-1 and IGF-II) to the IGF-II/mannose 6-phosphate receptor in fetal rat myocardium Endocrinology 135 231-239.

2. Wang P H (1998) Insulin-like growth factor 1 and the heart. In: Levin E R, Nadler J L (eds) Endocrinology of Cardiovascular Function. Boston: Kluwer Academic Publishers 225-234.

3. Donath M Y Zapf J Eppenberger-Eberhardt M Froesch E R Eppenberger H M (1994) Insulin-like growth factor I stimulates myofibril development and decreases smooth muscle alpha-actin of adult cardiomyocytes Proc Natl Acad Sci USA 91 1686-1690.

4. Fuller J Mynett J R Sugden P H (1992) Stimulation of cardiac protein synthesis by insulin-like growth factors Biochem J 282 85-90.

5. Ito H Iroe M Hirata Y Tsujino M Adachi S Schichiri M Koike A Nogami A Marumo F (1993) Insulin-like growth factor-I induces hypertrophy with enhanced expression of muscle specific genes in cultured rat cardiomyocytes Circulation 87 1715-1721.

6. Chen W H Pellegata N S Wang P H (1995) Coordinated effects of insulin-like growth factor I on inhibitory pathways of cell cycle progression in cultured cardiac muscle cells Endocrinology 136 40-44.

7. Reiss K Cheng W Ferber A Kajstura J Li P Li B Olivetti G Homcy C J Baserga R Anversa P (1996) Overexpression of insulin-like growth factor-1 in the heart is coupled with myocyte proliferation in transÍgenic mice Proc Natl Acad Sci USA 93 8630-8635.

8. Wang L Ma W Markovich R Chen J W Wang P H (1998) Regulation of cardiomyocyte apoptotic signaling by insulin-like growth factor I Circ Res 83 516-522.

9. Florini J R Ewton D Z Coolican S A (1996) Growth hormone and insulin-like growth factor system in myogenesis Endocrine Rev 15 481-517.

10. LeRoith D (1997) Insulin-like growth factors N Engl J Med 336 633-640.

11. Cittadini A Stromer H Katz S E Clark R Moses A C Morgan J P Douglas P S (1996) Differential cardiac effects of growth hormone and insulin-like growth factor-1 in the rat. A combined in vivo and in vitro evaluation Circulation 93 800-809.

12. Cittadini A Ishiguro Y Strömer H Spindler M Moses A C Clark R Douglas P S Ingwall J S Morgan J P (1998) Insulin-like growth factor-1 but not growth hormone augments mammalian myocardial contractility by sensitizing the myofilament to Ca²⁺through a wortmannin-sensitive pathway: studies in rat and ferret isolated muscles Circ Res 83 50-59.

13. MacLellan W R Schneider M D (1997) Death by design. Programmed cell death in cardiovascular biology and disease Circ Res 81 137-144.

14. Donath M Y Sutsch G Yan X-W Piva B Brunner H-P Glatz Y Zapf J Follath F Froesch E R Kiowski W (1998a) Acute cardiovascular effects of insulin-like growth factor I in patients with chronic heart failure J Clin Endocrinol Metab 83 3177-3183.

15. Donath M Y Jenni R Brunner H-P Anrig M Kohli S Glatz Y Froesch E R (1996) Cardiovascular and metabolic effects of insulin-like growth factor I at rest and during exercise in humans J Clin Endocrinol Metab 81 4089-4094.

16. Kolaczynski J W Caro J F (1994) Insulin-like growth factor-1 therapy in diabetes: physiologic basis, clinical benefits and risks Ann Intern Med 120 47-55.

17. Moses A C Young S C J Morrow L A O>>Brian M Clemmons D R (1996) Recombinant human insulin-like growth factor-I increases insulin sensitivity and improves glycemic control in type II diabetes Diabetes 45 91-100.

18. Freestone N S Ribaric S Mason W T (1996) The effect of insulin-like growth factor-1 on adult rat cardiac contractility Mol Cell Biochem 163/164 223-229.

19. Ren J Walsh M F Hamaty M Sowers J R Brown R A (1998) Altered inotropic response to insulin-like growth factor I in diabetic rat heart: influence of intracellular Ca²⁺and nitric oxide Am J Physiol 275 H823-H830.

20. Ren J Sowers J R Natavio M Brown R A (1999a) Influence of age on iontropic response to insulin and insulin-like growth factor I in spontaneously hypertensive rats: role of nitric oxide Proc Soc Exp Biol Med 221 46-52.

21. Guse A H Kiess W Funk B Kessler U Berg I Gercken G (1992) Identification and characterization of insulin-like growth factor receptors on adult rat cardiac myocytes: linkage to inositol 1, 4, 5-trisphosphate formation Endocrinology 130 145-151.

22. Sowers J R (1997) Insulin and insulin-like growth factor in normal and pathological cardiovascular physiology Hypertension 29 691-699.

23. Touyz R M Schiffrin E L (1997) Growth factors mediate intracellular signaling in vascular smooth muscle cells through protein kinase C-linked pathways Hypertension 30 1440-1447.

24. Piedras-Renteria E S Chen C C Best P M (1997) Antisense oligonucleotides against rat brain alpha1E DNA and its atrial homologue decrease T-type calcium current in atrial myocytes Proc Natl Acad Sci USA 94 14936-14941.

25. Solem M L Thomas A P (1998) Modulation of cardiac Ca²⁺channels by IGF1 Biochem Biophys Res Commun 252 151-155.

26. Guo W Kada K Kamiya K Toyama J (1997) IGF-I regulates K⁺-channel expression of cultured neonatal rat ventricular myocytes Am J Physiol 272 H2599-H25606.

27. Guo W Kamiya K Yasui K Kodama I Toyama J (1998) 1-adrenoceptor agonists and IGF-1, myocardial hypertrophic factors, regulate the Kv1.5 K⁺channel expression differentially in cultured newborn rat ventricular cells Pflügers Arch 436 26-32.

28. Buerke M Murohara T Skurk C Nuss C Tomaselli K Lefer A M (1995) Cardioprotective effect of insulin-like growth factor I in myocardial ischemia followed by reperfusion Proc Natl Acad Sci USA 92 8031-8035.

29. Li Q Li B Wang X Leri A Jana K P Liu Y Kajstura J Baserga R Anversa P (1997) Overexpression of insulin-like growth factor-1 in mice protects from myocyte death after infarction, attenuating ventricular dilation, wall stress, and cardiac hypertrophy J Clin Invest 100 1991-1999.

30. Parrizas M LeRoith D (1997) Insulin-like growth factor-1 inhibition of apoptosis is associated with increased expression of the bcl-xL gene product Endocrinology 138 1355-1358.

31. O'Connor R Kauffmann-Zeh A Liu Y Lehar S Evan GI Baserga R Blattler W A (1997) Identification of domains of the insulin-like growth factor I receptors that are required for protection from apoptosis Mol Cell Biol 17 427-435.

1. A method for treating heart failure, the method comprising the steps of:
a) providing a bioactive agent comprising one or more sequences derived from human IGF-1, including analogues and functional portions thereof,
b) incorporating the bioactive agent into a vehicle to provide a deliverable composition,
c) delivering the composition to the heart in an intrapericardial manner in order to achieve a therapeutic effect.

2. A method according to claim 1, wherein the bioactive agent comprises a sequence selected from the group consisting of SEQUENCE ID'S 1-5 and analogues thereof.

3. A method according to claim 1, wherein the vehicle comprises a hydrogel.

4. A method according to claim 1, wherein treatment is accomplished in a preventive, curative, palliative, supportive, and/or restorative manner and delivery is accomplished by the use of a pericardial access device.

5. A method according to claim 1, wherein the bioactive agent comprises a sequence selected from the group consisting of SEQUENCE ID'S 1-5 and analogues thereof, the vehicle comprises a hydrogel, and delivery is accomplished by the use of a pericardial access device.

6. A method according to claim 5 wherein the composition is provided in a form selected from the group consisting of aqueous isotonic solutions, suspensions, gels, and bioactive impregnated polymers.

7. A method according to claim 6 wherein the IGF is obtained by a method selected from the group consisting of isolation from a naturally-occurring source, chemical synthesis, and production by recombinant DNA technology.

8. A method according to claim 5 wherein the access device is selected from the group consisting of percutaneous transventricular access devices and transthoracic access devices.

9. A method according to claim 5 wherein the composition is provided in a form selected from the group consisting of aqueous isotonic solutions, suspensions, gels, and bioactive impregnated polymers; the IGF is obtained by a method selected from the group consisting of isolation from a naturally-occurring source, chemical synthesis, and production by recombinant DNA technology; and the access device is selected from the group consisting of percutaneous transventricular access devices and transthoracic access devices.

10. A method according to claim 9 wherein the IGF-1 peptide putative peptide that comprises one or more unique regions of hIGF-1 which are not shared by insulin and IGF-II.

11. A deliverable composition comprising a bioactive agent comprising one or more sequences derived from human IGF-1 and a pharmaceutically acceptable vehicle, the composition being adapted for intrapericardial delivery to the heart.

12. A deliverable composition according to claim 11, wherein the bioactive agent comprises a sequence selected from the group consisting of SEQUENCE ID'S 1-5 and analogues thereof.

13. (canceled)

14. (canceled)

15. (canceled)

16. (canceled)

17. (canceled)

18. (canceled)

19. (canceled)

20. (canceled)

21. A system comprising a deliverable composition according to claim 11, together with a delivery device as described herein.

22. (canceled)

23. (canceled)

24. (canceled)

25. A system according to claim 21, wherein the bioactive agent comprises a sequence selected from the group consisting of SEQUENCE ID'S 1-5 and analogues thereof, the vehicle comprises a hydrogel, and delivery is accomplished by the use of a pericardial access device.

26. (canceled)

27. (canceled)

28. A system according to claim 25 wherein the access device is selected from the group consisting of percutaneous transventricular access devices and transthoracic access devices.

29. A system according to claim 25 wherein the composition is provided in a form selected from the group consisting of aqueous isotonic solutions, suspensions, gels, and bioactive impregnated polymers; the IGF is obtained by a method selected from the group consisting of isolation from a naturally-occurring source, chemical synthesis, and production by recombinant DNA technology; and the access device is selected from the group consisting of percutaneous transventricular access devices and transthoracic access devices.

30. (canceled)

31. A delivered composition comprising a deliverable composition according to claim 11 positioned in situ within the pericardial space of the heart.

32. A delivered composition according to claim 31, wherein the bioactive agent comprises a sequence selected from the group consisting of SEQUENCE ID'S 1-5 and analogues thereof.

33. (canceled)

34. (canceled)

35. (canceled)

36. (canceled)

37. (canceled)

38. (canceled)

39. (canceled)

40. (canceled)

41. A bioactive agent comprising an IGF-1 peptide comprising one or more unique regions of hIGF-1 which are not shared by insulin and IGF-II.

42. A bioactive agent according to claim 41, wherein the agent comprises a protein sequence selected from the group consisting of SEQUENCE ID Nos. 1-5.


SEQUENCE LISTING (12 sequences)

SEQ ID NO: 1 - protein, 49 aa, Artificial sequence
  Sequence is synthesized
GFYFNKPTGYGSSSRRAPQTGIVDECCFRSCDLRRLEMYCAPLKPAKSA

SEQ ID NO: 2 - protein, 41 aa, Artificial sequence
  Sequence is synthesized
GYGSSSRRAPQTGIVDECCFRSCDLRRLEMYCAPLKPAKSA

SEQ ID NO: 3 - protein, 20 aa, Artificial sequence
  Sequence is synthesized
GYGSSSRRAPQTPLKPAKSA

SEQ ID NO: 4 - protein, 60 aa, Artificial sequence
  Sequence is synthesized
GYGSSSRRAPQTPLKPAKSAGYGSSSRRAPQTPLKPAKSAGYGSSSRRAPQTPLKPAKSA

SEQ ID NO: 5 - protein, 20 aa, Artificial sequence
  Sequence is synthesized
PLKPAKSAGYGSSSRRAPQT

SEQ ID NO: 6 - protein, 21 aa, Artificial sequence
  Sequence is synthesized
MGKISSLPTQLFKCCFCDFLK

SEQ ID NO: 7 - protein, 27 aa, Artificial sequence
  Sequence is synthesized
VKMHTMSSSHLFYLALCLLTFTSSATA

SEQ ID NO: 8 - protein, 29 aa, Artificial sequence
  Sequence is synthesized
GPETLCGAELVDALQFVCGDRGFYFNKPT

SEQ ID NO: 9 - protein, 12 aa, Artificial sequence
  Sequence is synthesized
GYGSSSRRAPQT

SEQ ID NO: 10 - protein, 21 aa, Artificial sequence
  Sequence is synthesized
GIVDECCFRSCDLRRLEMYCA

SEQ ID NO: 11 - protein, 8 aa, Artificial sequence
  Sequence is synthesized
PLKPAKSA

SEQ ID NO: 12 - protein, 35 aa, Artificial sequence
  Sequence is synthesized
RSVRAQRHTDMPKTQKEVHLKNASRGSAGNKNYRM